(19) United States
(12) Patent Application Publication
(10) Pub. No.: US 2022/0288588 A1
(43) Pub. Date: Sep. 15, 2022

(54) MICROFLUIDIC PASSIVE PLASMA SEPARATION DEVICE AND METHOD

(71) Applicant: Auer Precision Company, Inc., Mesa, AZ (US)

(72) Inventors: Eric Sanders, Phoenix, AZ (US); Anna Wood, Queen Creek, AZ (US); Everett Reynolds, Mesa, AZ (US)

(73) Assignee: Auer Precision Company, Inc., Mesa, AZ (US)

(21) Appl. No.: 17/632,966

(22) PCT Filed: Aug. 5, 2020
(86) PCT No.: PCT/US2020/045051

§ 371 Date: Feb. 4, 2022

Related U.S. Application Data
(60) Provisional application No. 62/883,021, filed on Aug. 5, 2019.

(51) Int. Cl. B01L 3/00; A61M 1/34

(52) U.S. Cl. CPC B01L 3/502753; B01L 3/5635; A61M 1/34; B01L 2300/0681; B01L 2300/0887; B01L 2400/0406

(57) ABSTRACT

A microfluidic passive plasma separation device is disclosed that provides rapid and efficient separation of optically clear plasma from whole blood. In various embodiments, the device comprises an engineered filter pad; a microfluidic capillary channel; and a plasma collection reservoir in fluidic communication, wherein separated plasma flows by capillary forces from the filter to the reservoir until the plasma in the collection reservoir provides sufficient hydrostatic head pressure to overcome the capillary forces and stop the separation even in order to prevent contamination of the separated plasma with blood cells previously trapped in the filter.

### CROSS-REFERENCE TO RELATED APPLICATIONS

This application claims priority to and the benefit of U.S. Provisional Application Ser. No. 62/883,021 filed Aug. 5, 2019 and entitled “Microfluidic Passive Plasma Separation Device and Method,” the disclosure of which is incorporated herein by reference in its entirety for all purposes.

### FIELD

The present disclosure generally relates to medical and diagnostic devices, and in particular, to a microfluidic device and method for separating plasma from whole blood.

### BACKGROUND

Blood is a physiologic fluid consisting of many components, each having a particular use in the practice of medicine. The primary components of blood include plasma, red blood cells, white blood cells, and platelets. Typically, whole blood comprises about 55% plasma and 45% blood cells and platelets by volume. For various medical diagnoses, it is necessary to first separate the plasma from a whole blood sample and then perform the diagnostics on the plasma thus obtained. For example, many microfluidic point of care (POC) medical devices used today require plasma in order to function. The traditional method of separating plasma from whole blood is to simply centrifuge the sample and pipette the clear plasma off from the top of the sample in the centrifuge tube. The plasma thus separated offboard may then be pipetted onto a diagnostic device. The cellular materials, e.g., the white and red blood cells and platelets, also separate in the centrifuge tube, appearing as layers below the liquid plasma layer. In other instances, whole blood may be centrifuged directly onboard to a diagnostic device, using a pump or other active transport method.

Blood plasma is a complex component of whole blood. Although plasma comprises about 92-95% water by volume, the non-water constituents are a complicated mixture of dissolved carbohydrates, fats, proteins, clotting factors, DNA, hormones, electrolytes and other substances, some dependent on the nature and health of the particular individual. Blood serum is also a liquid fraction from blood, but is the liquid remaining after whole blood is allowed to clot. Plasma, on the other hand, is the liquid fraction obtained from a whole blood sample prevented from clotting, e.g., by prior addition of an anticoagulant to the blood sample.

Hemolysis is the rupture of the membrane surrounding a blood cell. Red blood cells are very easy to rupture, and the expelled intracellular materials upon rupture can contaminate plasma as it is mechanically separated from whole blood. This is particularly a nuisance in centrifugation, where the red blood cells are mechanically ruptured, contaminating the plasma layer with hemoglobin, lactoferrin and other undesirables as the plasma separates in the centrifuge tube. Coloration of the plasma sample is a visual indication of erythrocyte hemolysis, and many procedures have been developed detailing proper handling of blood samples and optimal times and speed of centrifugation in order to mitigate hemolysis.

A centrifuge, even when benchtop in size, is bulky and expensive and not practical for POC diagnostics or even for use in small clinics. Further, centrifugation offboard or even onboard to a diagnostic device adds complexity and cost. Given these and other shortcomings, new devices, such as small or even microfluidic devices, are needed that can rapidly, efficiently and cost effectively separate plasma from whole blood with little or no hemolysis of the blood cells.

### SUMMARY

In various embodiments, a microfluidic passive plasma separation device for the rapid and efficient separation of plasma from whole blood is disclosed. The device operates entirely passively, i.e., by capillary action, without the need for mechanical pumps or other means to push or pull the blood through the separation process, and is thus simple and cost effective to manufacture and use.

In various embodiments, a microfluidic passive plasma separation device comprises an engineered filter pad having an inlet and an outlet; at least one microfluidic capillary channel in fluidic communication with the outlet of the engineered filter pad; and a plasma collection reservoir in fluidic communication with the at least one microfluidic capillary channel.

Broadly generalized, and in no way meant to limit the scope of the present disclosure, the engineered filter pad is configured to separate plasma from a whole blood sample, and the at least one microfluidic capillary channel is configured to move the separated plasma from the outlet of the engineered filter pad to the plasma collection reservoir. The microfluidic capillary channel acts as a capillary pump, creating a lower pressure at the outlet of the engineered filter pad that accelerates the blood separation therethrough.

In various embodiments, a microfluidic passive plasma separation device herein comprises a multilayer laminated structure. Each of the various laminated layers in the device may, for example, be configured to (a) structurally stabilize the overall device, e.g., to keep it from bending in use or warping over time, (b) positionally locate and hold in place an inner feature, such as the engineered filter pad, (c) provide for the at least one microfluidic capillary channel, (d) provide an opening to the device and/or a defined application site for the whole blood sample, (e) provide adhesion between two adjacent layers, and/or (f) dimensionally define a volume for the plasma collection reservoir in which the separated plasma may collect.

For example, a bottom layer of a microfluidic plasma separation device comprising a laminate structure may function to seal off the bottom of the device and prevent any fluid flow (i.e., leakage) out the bottom of the device, and/or to provide rigidity to the device. In another non-limiting example, one or more layers may be cut or bored through to provide the at least one microfluidic capillary channel. In still other examples, one or more layers laminated together may define the cross-sectional outline of the microfluidic capillary channel or the physical boundaries of the plasma collection reservoir, and hence the volumetric capacity of the reservoir.

In various embodiments, the microfluidic passive plasma separation device comprises an engineered filter pad. The filter pad may be situated in a locating feature provided in one layer of a multilayer laminate structure. For example, the engineered filter pad may be located within a recess or in an opening configured in a layer, and/or disposed and held between two layers of the laminate structure. In various embodiments, the engineered filter pad comprises one substantially homogenous material. In other embodiments, the engineered filter pad may comprise more than one filter layer arranged in series. The engineered filter pad is capable of filtering whole blood, and in particular, capable of providing an output of plasma from an input of whole blood. The engineered filter pad is designed to trap and hold back red and white blood cells and blood platelets. In various embodiments, the engineered filter pad acts as a membrane system capable of separating plasma from whole blood by capillary action through the filter material of the engineered filter pad. Other mechanical aspects of the separation device, e.g., the microfluidic capillary channel, are designed to optimize the efficiency of the engineered filter pad.

In various embodiments, the engineered filter pad comprises a homogeneous filtration material capable of retaining white blood cells, red blood cells and platelets. The liquid material able to pass through the engineered filter pad and exit therefrom comprises separated blood plasma. The blood plasma thus separated is found not to be contaminated by white or red blood cells or platelets, or by any intracellular materials that may have resulted from hemolysis of cells. The separated plasma may move to various locations in the device, such as to a plasma collection reservoir, by at least one capillary channel configured in the device to enable capillary action.

In various embodiments, the microfluidic passive plasma separation device comprises a laminated stack of five structural layers; and an engineered filter pad, for a total of six elements. The engineered filter pad may be located in a cut away portion of one of the layers, and held in place within the laminate by two other laminate layers, one above and one below the layer with the filter pad fitted therein. One or more layers may also provide at least one microfluidic capillary channel for the plasma to move from the outlet of the engineered filter pad to the plasma collection reservoir in the device. In various embodiments, at least one specially cut microfluidic capillary channel in one or more of the layers of the laminated device create a capillary flow for the separated plasma that, in effect, speeds up separation of blood through the engineered filter pad and increases the overall rate of separation of plasma.

In various embodiments, the engineered filter pad is chemically treated, such as to enhance separation rates or provide additional benefits to the device and its operation. In various examples, these coatings may ensure the surfaces in the filter pad are hydrophilic. In various embodiments, chemical treatment eliminates or at least mitigates analyte binding. In various examples, chemical treatment mitigates clotting of the blood sample as it passes through the engineered filter pad.

In various embodiments, the engineered filter pad of the microfluidic passive plasma separation device may be chemically treated with any combination of surfactant, calibrated isotonic buffers, hydrophilic coating matrix or agglutinating agents.

In various embodiments, a microfluidic passive plasma separation device is integrated directly to a point of care (POC) microfluidic diagnostic device. In such integrated devices, plasma thus separated in the microfluidic passive plasma separation device is directly channeled, such as by capillary flow, to a diagnostic device placed in fluidic communication with the microfluidic passive plasma separation device, rather than into a reservoir of the microfluidic passive plasma separation device.

In various embodiments, a plurality of microfluidic passive plasma separation devices may be laminated on a single backing card, making manufacturing faster and more efficient. In various embodiments, the multidevice cards can be cut apart to provide individual devices that can be packaged as appropriate for marketing and sales. In other embodiments, a card comprising a plurality of microfluidic passive plasma separation devices may be used as is, wherein a plurality of pipettes manually or robotically apply a drop of whole blood to each inlet port at the same time.

In various embodiments, a microfluidic passive plasma separation device comprises a multilayered structure.

In various embodiments, a multilayered microfluidic device comprises: an engineered filter pad having an inlet and an outlet, the engineered filter pad capable of separating plasma from whole blood; a plurality of plasma collection channels in fluidic communication with the outlet of the engineered filter pad; a first microfluidic capillary channel having a first end and a second end, the first end of the first microfluidic capillary channel in fluidic communication with the plurality of plasma collection channels; and a plasma collection reservoir in fluidic communication with the second end of the first microfluidic capillary channel, the plasma collection reservoir configured to collect separated plasma; wherein the plurality of plasma collection channels, the first microfluidic capillary channel and a portion of the plasma collection reservoir are configured in a single layer of the multilayered microfluidic device.

In various embodiments, the multilayered microfluidic device further comprises a blood application port.

In various embodiments, the blood application port and the engineered filter pad are configured on separate layers of the multilayered microfluidic device.

In various embodiments, the blood application port is configured in a first layer, and wherein the engineered filter pad is positioned in a second layer adjacent the first layer such that the blood application port is in fluidic communication with the inlet of the engineered filter pad.

In various embodiments, the blood application port is configured on a layer distinct from the single layer containing the plurality of plasma collection channels, the first microfluidic capillary channel, and a portion of the plasma collection reservoir.

In various embodiments, the engineered filter pad is disposed between the blood application port and the plurality of plasma collection channels, and is in fluidic communication with the blood application port and the plurality of plasma collection channels.

In various embodiments, the inlet of the engineered filter pad and the plurality of plasma collection channels are spatially arranged in the multilayered microfluidic passive plasma separation device to promote both lateral and vertical flow of a blood sample through the engineered filter pad.

In various embodiments, the first microfluidic capillary channel comprises a narrow portion having a first length and a first width that transitions into a wide portion having a second length and a second width, the narrow portion in fluidic communication with the plurality of plasma collection channels and the wide portion in fluidic communication with the plasma collection reservoir.

In various embodiments, the plurality of plasma collection channels, the first microfluidic capillary channel, and the plasma collection reservoir are each configured to collectively provide a capillary pump sufficient to pull separated plasma through the engineered filter pad and convey the separated plasma from the outlet of the engineered filter pad to the plasma collection reservoir.

In various embodiments, a portion of the plasma collection reservoir and the outlet of the engineered filter pad are spatially configured above the first microfluidic capillary channel such that plasma collected in the plasma collection reservoir can provide a sufficient hydrostatic head pressure to overcome the capillary pump.

In various embodiments, the multilayered microfluidic device further comprises an air vent fluidically connected to the plasma collection reservoir by a second microfluidic capillary channel such that the outlet of the engineered filter pad, the plurality of plasma collection channels, the first microfluidic capillary channel, the plasma collection reservoir, the second microfluidic capillary channel, and the air vent are fluidically connected to one another in series.

In various embodiments, the plurality of plasma collection channels, the first microfluidic capillary channel, the plasma collection reservoir, the second microfluidic capillary channel, and the air vent are each configured to collectively provide a capillary pump sufficient to pull separated plasma through the engineered filter pad and convey the separated plasma from the outlet of the engineered filter pad to the plasma collection reservoir.

In various embodiments, a portion of the plasma collection reservoir and the outlet of the engineered filter pad are spatially configured above the first microfluidic capillary channel such that plasma collected in the plasma collection reservoir can provide a sufficient hydrostatic head pressure to overcome the capillary pump.

In various embodiments, the plurality of plasma collection channels comprises a dendritic structure further comprising a plurality of branches and a single main channel, wherein each branch converges into the single main channel that fluidically connects to the first end of the first microfluidic capillary channel.

In various embodiments, the engineered filter pad comprises a homogeneous matrix of borosilicate glass microfibers coated with an agglutinating agent.

In various embodiments, the engineered filter pad has an average pore size of 0.006 mm (6 μm) to about 0.01 mm (10 μm).

In various embodiments, a method for separating plasma from a blood sample comprises: disposing a blood sample at an inlet of an engineered filter pad having an inlet and outlet, the engineered filter pad configured to provide separated plasma at the outlet from a blood sample disposed at the inlet; separating plasma through the engineered filter pad to the outlet of the engineered filter pad; pulling the separated plasma from the outlet of the engineered filter pad into a plurality of plasma collection channels and conveying the separated plasma from the plurality of collection channels through a microfluidic capillary channel to a plasma collection reservoir by capillary forces; and collecting the separated plasma as a liquid in the plasma collection reservoir; wherein the plurality of plasma collection channels is in fluidic communication with the outlet of the engineered filter pad, the microfluidic capillary channel comprises a first end and a second end, and wherein the first end of the microfluidic capillary channel is in fluidic communication with the plurality of plasma collection channels and the second end of the microfluidic capillary channel is in fluidic communication with the plasma collection reservoir.

In various embodiments, the step of collecting the separated plasma as a liquid in the plasma collection reservoir results in formation of a vertical column of liquid plasma that provides a hydrostatic head of sufficient pressure to overcome the capillary forces and cease plasma separation through the engineered filter pad.

In various embodiments, a portion of the plasma collection reservoir and the outlet of the engineered filter pad are spatially configured above the microfluidic capillary channel such that the separated plasma collected in the plasma collection reservoir can provide a hydrostatic head of sufficient pressure to overcome the capillary forces.

In various embodiments, about 24 μL of optically clear plasma can be obtained from an initial whole blood sample measuring about 120 μL in the course of about 60 to about 90 seconds through a microfluidic passive plasma separation device in accordance with the present disclosure.

### BRIEF DESCRIPTION OF THE DRAWING FIGURES

The subject matter of the present disclosure is pointed out with particularity, and claimed distinctly in the concluding portion of the specification. A more complete understanding of the present disclosure, however, may best be obtained by referring to the detailed description and claims when considered in connection with the following drawing figures:

FIG. 1 illustrates a perspective view of a microfluidic passive plasma separation device in accordance with various embodiments of the present disclosure;

FIG. 2 illustrates an exploded view of a microfluidic passive plasma separation device in accordance with various embodiments of the present disclosure, wherein the device comprises a multilayered structure;

FIG. 3 illustrates a top view of a microfluidic passive plasma separation device in accordance with various embodiments of the present disclosure;

FIG. 4 illustrates a cross-sectional view of a microfluidic passive plasma separation device in accordance with various embodiments of the present disclosure, with the cross-section taken through the device in the center of a plasma collection reservoir configured in the device as indicated in FIG. 3;

FIG. 5 illustrates a cross-sectional view of a microfluidic passive plasma separation device in accordance with various embodiments of the present disclosure, with the cross-section taken through the device in the center of an air vent configured in the device as indicated in FIG. 3;

FIG. 6 illustrates a cross-sectional view of a microfluidic passive plasma separation device in accordance with various embodiments of the present disclosure, with the cross-section taken through the length of the device as indicated in FIG. 3;

FIG. 7 illustrates details of a fourth layer of a microfluidic passive plasma separation device in accordance with various embodiments of the present disclosure, wherein a plurality of collection channels having a dendritic structure converge and fluidically connect to a first microfluidic capillary channel in the device; and

FIGS. 8A and 8B illustrate two configurations for a plurality of plasma collection channels having a dendritic structure in accordance with various embodiments of the present disclosure.

### DETAILED DESCRIPTION

The detailed description of exemplary embodiments references the accompanying drawings, which show exemplary embodiments by way of illustration and their best mode. While these exemplary embodiments are described in sufficient detail to enable those skilled in the art to practice the invention, it should be understood that other embodiments may be realized and that logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

In various embodiments of the present disclosure, a microfluidic passive plasma separation device is described. In various embodiments, the microfluidic passive plasma separation device provides rapid and efficient separation of plasma from whole blood.

In various embodiments, a microfluidic passive plasma separation device comprises an engineered filter pad having an inlet and an outlet; at least one microfluidic capillary channel in fluidic communication with the outlet of the engineered filter pad; and a plasma collection reservoir in fluidic communication with the at least one microfluidic capillary channel.

In various embodiments, a microfluidic passive plasma separation device integrated with a POC diagnostic assay provides rapid analysis of a blood sample, the sample is physically controlled and contained once absorbed into the microfluidic passive plasma separation device. In various embodiments, the overall geometry of the microfluidic passive plasma separation device can be optimized to integrate directly to a POC diagnostic assay.

In various embodiments, a method for separating plasma from whole blood is described. In various embodiments, the method comprises disposing a whole blood sample at an inlet of an engineered filter pad having an inlet and an outlet, the engineered filter pad capable of separating plasma from whole blood; pulling separated plasma through the engineered filter pad; and conveying separated plasma from the outlet of the engineered filter pad through at least one microfluidic capillary channel to a plasma collection reservoir in fluidic communication with the at least one microfluidic capillary channel.

### Definitions and Interpretations

As used herein, the term “whole blood” or “whole blood sample” refers generally to blood as obtained from any human individual or any non-human animal subject. In various embodiments, a whole blood sample is virgin and neat, i.e., in its native form as extracted from the individual or animal subject. Herein, whole blood thus obtained may be subsequently modified with clotting factors, anticoagulants, agglutinating agents, or other substances as necessary to prepare the whole blood sample for separation of plasma.

As used herein, the term “anticoagulant” refers to any and all known factors added to whole blood to mitigate clotting. These factors include, for example, ethylenediaminetetraacetic acid and its various salts (EDTA), sodium citrate, theophylline, adenosine, dipyridamole, heparin and its various salts, sodium fluoride, and acid citrate d-dextrose. The anticoagulant may already be incorporated in a blood collection tube, e.g., a “sodium citrate tube,” “CTAD tube” (citrate, theophylline, adenosine and dipyridamole), a “lithium heparin tube,” or a “sodium heparin tube,” and so forth, making separate addition of an anticoagulant unnecessary since the anticoagulant is mixed with the whole blood as it is drawn into the appropriate tube. An anticoagulant is used when plasma is the desired result from the fluidic device and separation method disclosed herein.

As used herein, the term “clotting factor” or “clot activator” refers to any and all known factors added to whole blood to promote clotting. These factors include, for example, silica, calcium and thrombin. A clotting factor may be used when serum is the desired result from a fluidic device, recognizing that whole blood naturally clots even in the absence of added substances.

As used herein, the term “agglutinating agent” refers to any substance known to affect agglutination of red or white blood cells. Such regents include, but are not limited to, anti-A and anti-B IgM antibodies that interact with corresponding antigens at the surfaces of red blood cells leading to agglutination of the cells. As used herein, an agglutinating agent may be adsorbed into and dried within the interstices of the engineered filter pad such that blood cells can undergo agglutination as the whole blood sample is absorbed into the engineered filter pad and contacts the impregnated agglutinating agent. In other embodiments, an agglutination agent may be added to a whole blood sample before the blood sample is applied to a microfluidic passive plasma separation device of the present disclosure.

As used herein, the term “fluidic communication” means that two elements are connected together in such a way that liquid may flow between the two elements, e.g., in one direction or in both directions. The term fluidic communication is not meant to exclude an intervening element. So, for example, element A and element B may be in fluidic communication, but in more specific embodiments, an element C may be configured between A and B such that elements A, B and C are in fluidic communication, being arranged in series A-C-B.

As used herein, the term “inlet,” used in conjunction with an engineered filter pad, refers to the side or end of the engineered filter pad positioned to receive a sample of whole blood for plasma separation. As used herein, the term “outlet,” used in conjunction with an engineered filter pad, refers to the side or end of the engineered filter pad from which separated plasma emanates when a sample of whole blood is provided at the opposite, inlet side or end. In various embodiments, an engineered filter pad for use herein, separate from any other features of a microfluidic passive plasma separation device, may be symmetrical in both physical appearance and function. That is, separation of plasma may be possible from whole blood traveling in either direction through the filter pad. For example, a commercially available engineered filter pad for separation of blood plasma from whole blood may be in the shape of a disc, with the two opposing circular sides of the disc visually indistinguishable and capable of functioning as the inlet or the outlet of the filter pad. Therefore, the terms “inlet” and “outlet” in conjunction with an engineered filter pad, refer to the two opposite sides or the two opposite ends (depending on physical shape and orientation) of the filter pad when the engineered filter pad is positioned within a microfluidic passive plasma separation device herein. In general, the inlet of the engineered filter pad, when part of a microfluidic passive plasma separation device, will be accessible, so that a sample of whole blood can be disposed on it. On the other hand, the outlet of the engineered filter pad will necessarily reside inside the confines of the microfluidic passive plasma separation device and will generally not be accessible so as to maintain the integrity of the sample. In other embodiments, an engineered filter pad may not be functionally symmetrical, and the filter pad may have clearly marked inlet and outlet faces or edges so that it can be oriented correctly in a microfluidic passive plasma separation device. Such asymmetrical filters may comprise inhomogeneous arrangements of filter materials, e.g., providing a gradation of pore size, mandating that blood flow be only in one direction through the filter for proper separation.

As used herein, the term “capillary channel” refers to a capillary in a broad sense, so as not to imply a particular cross-sectional shape. A capillary is commonly known across many sciences to be a tube having a small circular cross-section with a diameter that can be an order of magnitude smaller than the overall length of the tube. For purposes herein, a capillary channel is not limited to a tube configuration. A capillary channel herein may have any cross-sectional shape, such as, but not limited to, triangular, rectangular, square, oval or circular. A capillary channel herein may comprise a boxed-in structure, such as comprising three layers of material that in combination define a capillary channel. In various embodiments, the middle layer of a three layer laminate may provide the spacers necessary to define the two long sides of a square or rectangular cross-section capillary, while the remaining two layers provide the top and the bottom of the channel. For purposes herein, a capillary channel is any fluidic conduit having any cross-sectional shape and length capable of capillary flow. The capillary pump principle is discussed, for example, in W. Guo, et al., “Capillary pumping independent of the liquid surface energy and viscosity,”Microsystems & Nanoengineering, 4, 2 (2018).

As used herein, reference to the “top” or “top face” and “bottom” or “bottom face” of a microfluidic passive plasma separation device herein refers to the two faces of the device when the device is lying flat on a surface in a position that allows use of the device for plasma separation. In various embodiments, a microfluidic passive plasma separation device herein is shaped like a long narrow strip, e.g., like a microscope slide or an elongated version thereof. Therefore, the length of a microfluidic passive plasma separation device herein is from about two to about five or six times the width, and the thickness of the device is merely fractional compared to the width. The top face of the device comprises an inlet for receipt of a blood sample, along with a plasma collection reservoir that is open to the top face to allow plasma retrieval. The bottom face of the device is the opposite side, and that face would be resting on a surface such as a laboratory countertop when the device is in use.

As used herein, reference to “vertical,” latitudinally,” and “longitudinally” refer to directions in a microfluidic passive plasma separation device of the present disclosure when the device resembles a microscope slide, i.e., a long, thin strip. Vertical refers to the direction through the thickness of the strip-shaped device. Latitudinally refers to the direction across the width of the device, and longitudinally refers t the direction down the length of the device.

As used herein, the term “multilayered” refers to a microfluidic passive plasma separation device herein having at least two layers rather than being machined from a single piece of material like a flat thin strip of plastic. In various embodiments, a microfluidic passive plasma separation device according to the present disclosure comprises 2 layers, 3 layers, 4 layers, 5 layers, 6 layers, 7 layers, 8 layers, 9 layers, or 10 layers or more. In various embodiments, a microfluidic passive plasma separation device herein comprises a multilayered construct wherein each of the layers have substantially identical overall length and width dimensions such that a stack of layers can be aligned and then bonded together. In various embodiments, a layer may be comprised of two or more layers. In other words, any layer within a multilayered microfluidic passive plasma separation device herein may comprise two or more sublayers. Layers and sublayers herein may comprise plastic films, plastic films treated on one or both sides such as to be more hydrophilic, plastic films coated with adhesive on one or both sides, one-sided adhesive tapes, two-sided adhesive tapes, laminates of plastic film and adhesive tape, and so forth. In various embodiments, an adhesive layer may be provided with a release liner to aid handling and use. Release liners may be removed prior to lamination of a multilayered device, or the release liner may not be removed prior to lamination and is integrated into the device as another layer for some purpose. The directional term “vertical,” in conjunction with a multilayered construct, refers to an orientation through the layers in a direction orthogonal to the flat surface of any one layer (i.e., vertical is the z-direction with the x/y plane being a top or bottom flat surface of a layer in the multilayered device). Similarly, reference to one element being disposed “above” or “below” another element means that the two elements are situated in relative to one another along the z-axis as defined.

### Microfluidic Passive Plasma Separation Device

In various aspects of the present disclosure, a microfluidic passive plasma separation device comprises an engineered filter pad having an inlet and an outlet; at least one microfluidic capillary channel in fluidic communication with the outlet of the engineered filter pad; and a plasma collection reservoir in fluidic communication with the at least one microfluidic capillary channel. Stated another way, a microfluidic passive plasma separation device comprises at least one microfluidic capillary channel fluidically connecting an outlet of an engineered filter pad to a plasma collection reservoir.

In various embodiments, a microfluidic passive plasma separation device comprises a machined block of material. In these embodiments, a small piece of polyester, polyethylene, polyester, polycarbonate or other plastic, such as in the shape of a microscope slide, is machined to provide apertures and channels.

In various embodiments, a microfluidic passive plasma separation device comprises a multilayered structure, wherein the number of layers total at least two. In a multilayered configuration, any single layer may comprise sublayers. A multilayered structure allows for the spatial offsetting of features vertically and/or horizontally. For example, the engineered filter pad may be disposed vertically above the plurality of plasma collection channels.

In various embodiments, a microfluidic passive plasma separation device comprises an engineered filter pad having an inlet and an outlet; a first microfluidic capillary channel having a first end and a second end, the first end of the first microfluidic capillary channel in fluidic communication with the outlet of the engineered filter pad; and a plasma collection reservoir in fluidic communication with the second end of the first microfluidic capillary channel.

In various embodiments, the engineered filter pad is configured to separate plasma from whole blood with the separated plasma transferring to the plasma collection reservoir and the blood cells and platelets remaining occluded in the engineered filter pad.

In various embodiments, the plasma collection reservoir is dimensionally configured as a well that is open at the top to an exterior environment to allow insertion of a pipette to retrieve separated plasma collected in the well. In this regard, the plasma collection reservoir may initially function as an air vent for the first microfluidic capillary channel, with the plasma collection reservoir eventually filling with separated plasma as plasma separation takes place.

In various embodiments, the microfluidic passive plasma separation device further comprises an air vent configured at an end of a second microfluidic capillary channel fluidically connected to the plasma collection reservoir. In various embodiments, the first and second microfluidic capillary channels may be arranged colinearly, with the first and second microfluidic capillary channels arranged 180° apart on a centrally located plasma collection reservoir.

In various embodiments, the microfluidic passive plasma separation device further comprises a plurality of plasma collection channels in fluidic communication with both the outlet of the engineered filter pad and the first microfluidic capillary channel such that the plurality of plasma collection channels converge into a first end of the first microfluidic capillary channel. The fluidic contact between the outlet end or side of the engineered filter pad and the plurality of plasma collection channels provides a route for the separated plasma to collect and converge into a main branch that leads into the first microfluidic capillary channel.

In various embodiments, the first microfluidic capillary channel further comprises a first portion and a second portion, such as a narrow portion and a wider portion, respectively. In various embodiments, the narrower portion of the first microfluidic capillary channel is fluidically connected to the outlet of the engineered filter pad or to a main branch in a plurality of plasma collection channels disposed in fluidic communication with the outlet of the engineered filter pad. In either configuration, separated plasma first enters the narrow portion of the first microfluidic capillary channel and then flows into the wider portion of the microfluidic capillary channel on the way to the plasma collection reservoir. The lengths and widths of the narrow and wider portions of the first microfluidic capillary channel, and various ratios thereof, are critical to optimizing capillary draw on the outlet of the engineered filter pad.

In various embodiments, a multilayered microfluidic passive plasma separation device comprises 2, 3, 4, 5, 6, 7, 8, 9, or 10 or more individual layers that are vertically stacked and bonded together. Any single layer may comprise at least two sublayers. In various embodiments, the individual layers that made up a finished device may no longer be discernable, because the initial layers were very thin and are bonded together in the finished device.

In various embodiments, the multilayered microfluidic passive plasma separation device comprises a vertical stack of at least five layers bonded to one another in a multilayer laminate structure.

In various embodiments, one layer of the multilayered structure comprises features that define channels in the plurality of plasma collection channels, the first microfluidic capillary channel, and a portion of the plasma collection reservoir.

In various embodiments, the plurality of plasma collection channels, the first microfluidic capillary channel and the second microfluidic capillary channel are configured in a single layer of the multilayered structure, whereas the plasma collection reservoir and the air vent are configured in at least two layers of the multilayered structure.

In various embodiments, the first microfluidic capillary channel is configured as a longitudinal trough down a length of a single layer of the multilayered structure, wherein two additional adjacent layers, each disposed on opposite sides of the longitudinal trough, cap the trough on opposites open sides to provide the first microfluidic capillary channel having a boxed-in structure.

In various embodiments, the plasma collection reservoir is configured as concentric apertures disposed through each of at least two adjacent layers in the multilayered structure.

In various embodiments, an air vent is configured as concentric apertures disposed through each of at least two adjacent layers in the multilayered structure, wherein the air vent fluidically connects to the plasma collection reservoir via a second microfluidic capillary channel.

In various embodiments, the engineered filter pad is disposed within concentric apertures configured in each of at least two adjacent layers.

The present disclosure is not limited in any way to the examples illustrated in the drawing figures and discussed below. As explained in more detail below, the size, shape, length, volume and capillary flow rate of the at least one microfluidic capillary channel in the device, e.g., the first microfluidic capillary channel, and the volumetric capacity of the plasma collection reservoir and spatial orientation relative to the outlet of the engineered filter pad, determine the plasma separation rate and the purity of the plasma collected by controlling the rate and duration of the separation process. Most importantly, a microfluidic passive plasma separation device within the scope of the present disclosure is not limited by the number of layers that might be employed in a multilayered device structure. Layers may be added or subtracted as needed, and although the specific embodiments may show a five layer embodiment having first, second, third, fourth and fifth layers that are aligned in a stack and bonded together, a multilayered microfluidic passive plasma separation device herein comprises at least two layers and is thus not limited to the five layers exemplified. As discussed, any single layer within a multilayered microfluidic passive plasma separation device herein may comprise two or more sublayers.

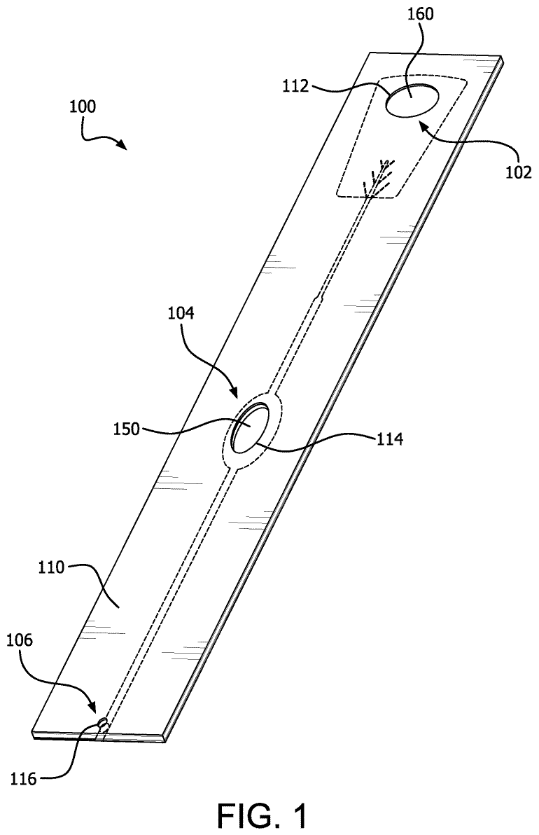

With reference now to FIG. 1, an embodiment of a microfluidic passive plasma separation device 100 in accordance with the present disclosure is illustrated. The device 100 is shown in a front perspective view. The device 100 comprises a blood application port 102 leading to and in fluidic communication with an engineered filter pad 160 underneath. As discussed in more detail herein, the blood application port is disposed above and in fluidic communication with the inlet of the engineered filter pad. Further, the port directs entry of a blood sample to a particular portion of the engineered filter pad. The blood application port 102 comprises a circular aperture 112 disposed in the top face 110 of the device, although the circular aperture is not meant to be limiting. As explained in more detail below, the top face 110 may comprise the first layer of a multilayer laminate structure. More than one aperture disposed in vertical alignment may make up the totality of the blood application port.

The microfluidic passive plasma separation device 100 further comprises a plasma collection reservoir 104. In various embodiments, the plasma collection reservoir 104 is dimensionally configured as a well open to the top of the device. The plasma collection reservoir 104 comprises an oval hole 114 disposed in the top face 110 of the device. Visible at the bottom of the plasma collection reservoir 104 is the bottom 150 of the device. As mentioned, the device may comprise a multilayer laminate structure, in which case the bottom 150 may comprise the bottommost layer of the multilayer laminate structure. Only a portion of the bottom layer 150 is visible through the aperture 114. Although illustrated as an oval opening, the shape of the hole 114 may be circular or any other shape. The plasma collection reservoir 104 transcends down to the bottom 150 and may comprise a cylindrical shape or an oval shape in cross-section.

With continued reference to FIG. 1, the dashed lines illustrate the underlying features in the device 100 that are not discernable from viewing the top face 110 of the device 100. The features, discussed below, include the engineered filter pad 160, a first microfluidic capillary channel having both a narrow portion and a wide portion leading to the plasma collection reservoir, and a second microfluidic capillary channel leading away from the plasma separation reservoir to an air vent 106.

In various embodiments, the device 100 further comprises an air vent 106 in fluidic communication with the plasma collection reservoir via a second microfluidic capillary channel. The air vent 106 further comprises a circular hole 116 configured in the top face 110 of the device 100, which may in some embodiments comprise the first layer of a multilayer laminate structure. As illustrated, three holes 112, 114 and 116 may be disposed in the top layer 110 of the device to provide for the blood application port 102, the plasma collection reservoir 104 and the air vent 106, respectively.

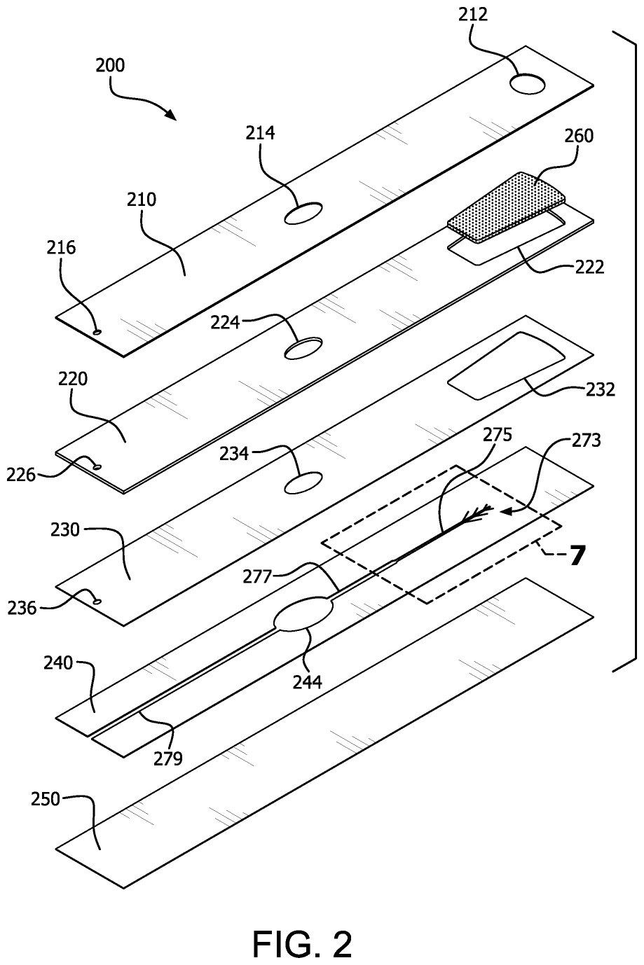

FIG. 2 illustrates embodiments of a microfluidic passive plasma separation device 200 in accordance with the present disclosure, comprising a multilayer laminate structure, shown here in an exploded view. As shown in FIG. 2, the microfluidic passive plasma separation device 200 comprises five layers disposed in the order of a first layer 210, a second layer 220, a third layer 230, a fourth layer 240, and a fifth layer 250, which are laminated together to form the working microfluidic passive plasma separation device 200. Each of these five layers are described in detail herein. It is evident from the drawing which face of which layer contacts the one or two adjacent layers when the five layers are laminated together as a composite structure. The microfluidic passive plasma separation device 200 comprises an engineered filter pad 260 that is trapped between the first layer 210 and the fourth layer 240 when the layers are laminated together. When the layers are laminated into a composite, various microfluidic capillary channels having a boxed-in structure are created that work in combination with the engineered filter pad 260.

In various embodiments, a microfluidic passive plasma separation device 200 measures from about 0.25 inches (6.35 mm) to about 2 inches (51 mm) in width by about 1 inch (25 mm) to about 6 inches (152 mm) in length, with a thickness of from about 10 mil (0.01 inch, 0.254 mm) to about 100 mil (0.1 inch, 2.54 mm). For example, a practical size for a microfluidic passive plasma separation device herein is 1 inch (25 mm) wide×6 inches (152 mm) long×65 mil (0.065 inch, 1.65 mm) thick. These dimensions are not meant to be limiting, and may be varying for such reasons as the size of the blood sample to be separated, and the end use of the microfluidic passive plasma separation device, such as if it is coupled to a diagnostic system that directly analyzes the separated plasma.

With continued reference to FIG. 2, the first (or top) layer 210 further comprises each of an aperture 212 that forms part of the blood application port (102 in FIG. 1), an aperture 214 that forms part of the plasma collection reservoir (104 in FIG. 1), and an aperture 216 that forms part of the air vent (106 in FIG. 1).

In various embodiments, first layer 210 comprises a cover, such as a sheet or film of plastic used to cap the microfluidic passive plasma separation device 200. In various embodiments, first layer 210 comprises a polyethylene terephthalate film (a polyester), a polycarbonate film, a polyethylene film, or a polypropylene film. In various embodiments, the first layer 210 comprises Melinex® or Mylar®, which are polyester (PET) films available from DuPont Teijin. In various embodiments, first layer 210 comprises from about 5 mil (0.005 inch, 0.127 mm) to about 15 mil (0.015 inch, 0.381 mm) thick PET film. Preferably a 10 mil (0.01 inch, 0.254 mm) thick Melinex® film is used for the top layer of the device.

With continued reference to FIG. 2, the microfluidic passive plasma separation device 200 comprises a second layer 220. Second layer 220 also includes three apertures disposed therethrough, namely aperture 222, configured in the approximate dimensions of the engineered filter pad 260, thus providing a recess for locating same, aperture 224 that forms part of the plasma collection reservoir (104 in FIG. 1), and an aperture 226 that forms part of the air vent (106 in FIG. 1).

In various embodiments, second layer 220 may comprise a plastic film layer, an adhesive layer, or a combination of the two in its own separate laminate structure comprising at least two layers. In various embodiments, second layer 220 comprises a laminate of plastic and adhesive film layers, wherein a central plastic film is coated on both sides with an adhesive, such as an acrylic adhesive. In various embodiments, second layer 220 comprises a polyester core layer coated on both sides with an acrylic adhesive. In specific embodiments, this layer may comprise 10 mil (0.01 inch, 0.254 mm) thick PET film coated on both sides with 2 mil (0.002 inch, 0.051 mm) to 5 mil (0.005 inch, 0.127 mm) thick acrylic adhesive layers. An exemplary second layer 220 is a combination of a 10 mil (0.01 inch, 0.254 mm) thick PET film (like Melinex® film) coated on both sides with acrylic adhesive for a total second layer 220 thickness of about 19 mil (0.019 inch, 0.48 mm). Alternatively, the 3M™ Membrane Switch Spacer 7959MP, available from 3M, Minneapolis, Minn., may be used as the second layer 220, which consists of 2.0 mil (0.002 inch, 0.051 mm) high-performance acrylic adhesive double coated on each side of a 5 mil (0.005 inch, 0.127 mm) thick polyester film carrier layer. With the aperture 222 and the acrylic adhesive layers, the second layer 220 locates the engineered filter pad 260 and bonds the first layer 210 to the third layer 230 by nature of its double-coated bonding tape structure.

In various embodiments, the microfluidic passive plasma separation device 200 comprises an engineered filter pad 260 for separating plasma from a sample of whole blood. The engineered filter pad 260 is designed to retain the red and white blood cells and the platelets present in whole blood and allow only the plasma and its dissolved constituents to pass through to move further along in the microfluidic passive plasma separation device 200. The material passing through the engineered filter pad 260 is optically clear plasma, in part because of the efficiency of the engineered filter pad 260, and also in part because the device automatically shuts itself off before the separated plasma can be compromised. As described in more detail below, if separated plasma is continually pulled out from the engineered filter pad 260 by capillary action, the red and white blood cells and platelets trapped in the engineered filter pad 260 will eventually dislodge and move through the microfluidic capillary channel to contaminate the separated plasma. One aspect of the microfluidic passive plasma separation device of the present disclosure is that it will cease to separate plasma when the plasma collection reservoir, and the microfluidic capillary channel leading to it, fill to capacity.

The engineered filter pad 260 may comprise borosilicate glass microfiber that is distributed substantially homogeneously throughout the volume of the filter. In this way, the glass microfiber acts as a homogenous filtration media or “separation membrane.” The materials in the filter pad 260 are hydrophilic, e.g., either intrinsically by nature of the materials or by virtue of hydrophilic surface treatment (chemical treatment, corona discharge, or other surface energy treatment, etc.).

In various embodiments, the average pore size of the engineered filter pad 260 is from about 0.006 mm (6 μm) to about 0.01 mm (10 μm). In various embodiments, the pore size is about 0.008 mm (8 μm). For fibrous media, the pores in the filter are not “holes” per se, but instead a complex network of pathways due to the random entanglement of borosilicate glass microfibers. The fibers within the engineered filter pad 260 may be treated with various acrylic binders to keep the fibrous filter structurally intact, and/or fibers may be treated with an agglutinating coating.

A suitable commercially available engineered filter pad 260 for use herein is the CF-D-23® coated whole blood separation media available from the I. W. Tremont Co., Inc., Hawthorne, N.J., USA. The “CF” in the product name indicates “cross-flow,” that is vertical flow, although as explained herein the positioning of the plurality of plasma collection channels under the engineered filter pad 260 can be shifted laterally to force both horizontal and vertical flow of plasma through the engineered filter pad 260. In various embodiments, a lateral flow engineered filter pad 260 may be used, such as the LF-D-23® coated whole blood separation media from I. W. Tremont. The “LF” in the product designation indicates “lateral flow.” This filter essentially comprises the same materials as the CF-D-23® coated whole blood separation media used as the engineered filter pad 10A in device 1A. The LF-D-23® filter pad, like the CF-D-23® filter pad, is coated with an agglutinating agent in order to improve plasma separation yield.

In various embodiments, the filtration material present in the engineered filter pad 260 is coated for high yield agglutination of the whole blood as it filters through, having basis weight of about 94.4 g/m², caliper thickness of 0.50 mm, micron retention of about 3-5 μm, and tensile strength of about 8.0 pounds in the machine direction/4.0 pounds in the cross-direction. The CF-D-23® or LF-D-23® coated whole blood separation media, for example, may be fitted into the laminated structure shown in FIG. 2, such as located within the aperture 222 disposed in the second layer 220, recognizing that the precise arrangement of laminated layers can be different from what is illustrated. In the device 200, the separated plasma emanating from the bottom outlet of the engineered filter pad 260 is pulled by capillary action to the plasma collection reservoir (104 in FIG. 1).

In various embodiments, the CF-D-23® or LF-D-23® coated whole blood separation media from I. W. Tremont, for example, provides a plasma yield of about 40% by volume when filtering a whole blood sample. In comparison, an engineered filter pad comprising borosilicate glass microfibers absent any agglutinating coating demonstrates a plasma yield of only about 15% by volume. However, it is important to incorporate the engineered filter pad 260, such as the CF-D-23® or LF-D-23® media, within a laminated device 200 or variation thereof in accordance with the present disclosure in order to increase the separation rate through the engineered filter pad 260, which would otherwise be unusably slow. Further, the design of the microfluidic capillary channels, the plasma collection reservoir, and other internal features of the microfluidic passive plasma separation device 200, are essential to ensure the device is self-limiting, i.e., that the device can shut itself off before the blood cells and platelets held back inside the interstices of the engineered filter pad 260 come out, which would be the case if the separation process continues for too long, (e.g., greater than about 90 seconds).

In various embodiments, the engineered filter pad 260 may be from about 10 mil (0.01 inch, 0.254 mm) to about 25 mil (0.025 inch, 0.635 mm) in thickness (i.e., the thickness through which plasma separates from a blood sample). In more specific examples, the engineered filter pad 260 may comprise about 0.3 in² (194 mm²) of surface area on both the symmetrical inlet and outlet faces, a tapering width (such as illustrated in the figures) from 0.44 inches (11 mm) across at the widest width to 0.30 inches (7.6 mm) across at the narrowest width, a length of about 0.8 inches (20 mm), and a thickness of about 26 mil (0.026 inches, 0.66 mm). In various embodiments, the engineered filter pad 260 may have a volume of about 0.008 in³ (131 mm³).

With continued reference to FIG. 2, the microfluidic passive plasma separation device 200 comprises a third layer 230 within the five layer laminate structure. As per second layer 220, the third layer 230 includes three apertures disposed therethrough, namely aperture 232, configured in the approximate dimensions of the engineered filter pad 260, thus providing a recess (in combination with aperture 222) for locating same, aperture 234 that forms part of the plasma collection reservoir (104 in FIG. 1), and an aperture 236 that forms part of the air vent (106 in FIG. 1).

Per second layer 220, the third layer 230 may comprise a plastic film layer, an adhesive layer, or a combination of the two comprising a separate laminate structure. In various embodiments, third layer 230 comprises a pressure-sensitive adhesive layer. In various embodiments, third layer 230 comprises a plastic film, optionally coated on one or both sides with a hydrophilic treatment. In various embodiments, the third layer 230 comprises a laminate having a hydrophilic plastic film layer and a pressure-sensitive adhesive layer on one or both sides of the plastic film layer. In various embodiments, the third layer 230 may comprise a two-layer laminate structure that further comprises a polyester film layer coated at least on the bottom side with a hydrophilic treatment (i.e., the side that will directly contact the fourth layer 240), and covered with double-sided adhesive layer on the top side (i.e., the side that will directly contact the second layer 220). In various embodiments, the third layer 230 comprises a layer of double-sided adhesive tape, such as AR90445 from Adhesives Research, Glen Rock, Pa., adhered to one side of a 4 mil (0.004 inch, 0.1 mm) thick layer of clear hydrophilic polyester film, such as 3M™ 9969 Microfluidic Diagnostic Film available from 3M, Minneapolis, Minn., for a total thickness of the third layer 230 of about 6 mil (0.006 inch, 0.15 mm).

With continued reference to FIG. 2, the microfluidic passive plasma separation device 200 comprises a fourth layer 240 within the five layer laminate structure, which may be referred to as the channel layer because of the presence of longitudinal cuts through the layer that define the widths and lengths of one or more microfluidic capillary channels and other fluidic pathways and features. As illustrated, fourth layer 240 comprises a plurality of plasma collection channels 273, disposed in a dendritic configuration, a first microfluidic capillary channel 275/277 in fluidic communication with the plurality of plasma collection channels 273, and an aperture 244 in fluidic communication with the first microfluidic capillary channel 275/277. The aperture 244 defines the fourth portion of the plasma collection reservoir (104 in FIG. 1), and more specifically its widest portion located at the very bottom of the reservoir.

In various embodiments, the fourth layer 240 may further comprise a second microfluidic capillary channel 279 disposed from the aperture 244 in a direction opposite the plasma collection channels 273 to the end of the device. As illustrated, the second microfluidic channel may run off the edge of the fourth layer 240, or, as not illustrated, may end just short of the end of the fourth layer 240 provided that the end of the second microfluidic channel 279 lines up with apertures 216, 226 and 236 in the layers above such that the combination of the second microfluidic channel 279 and the apertures 216, 226 and 236 form an air vent mechanism comprising the vent hole 106 indicated in FIG. 1.

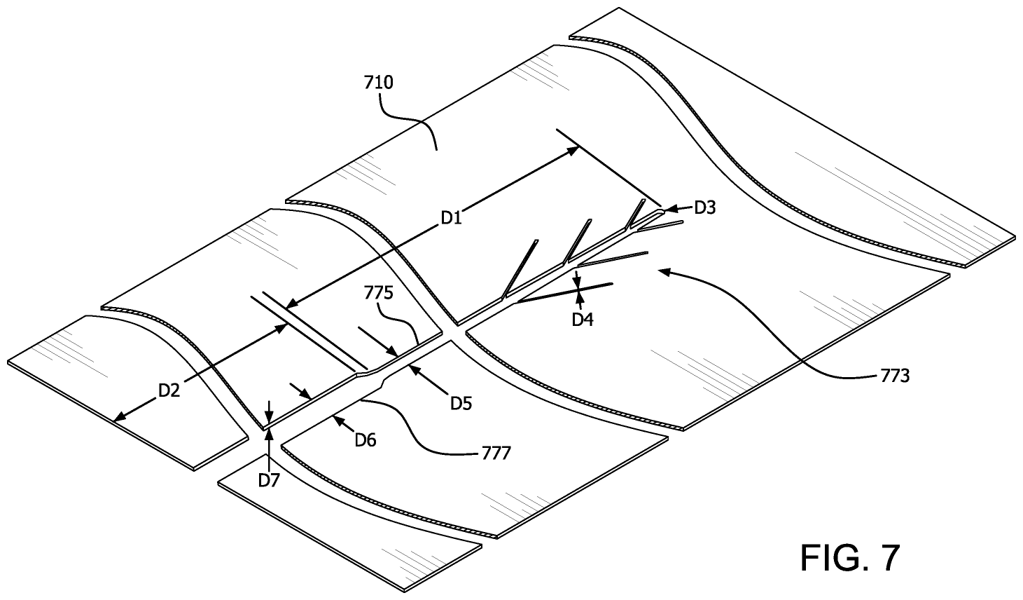

The dashed line box in FIG. 2 on the fourth layer 240 indicates the portion of the fourth layer 240 illustrated in FIG. 7.

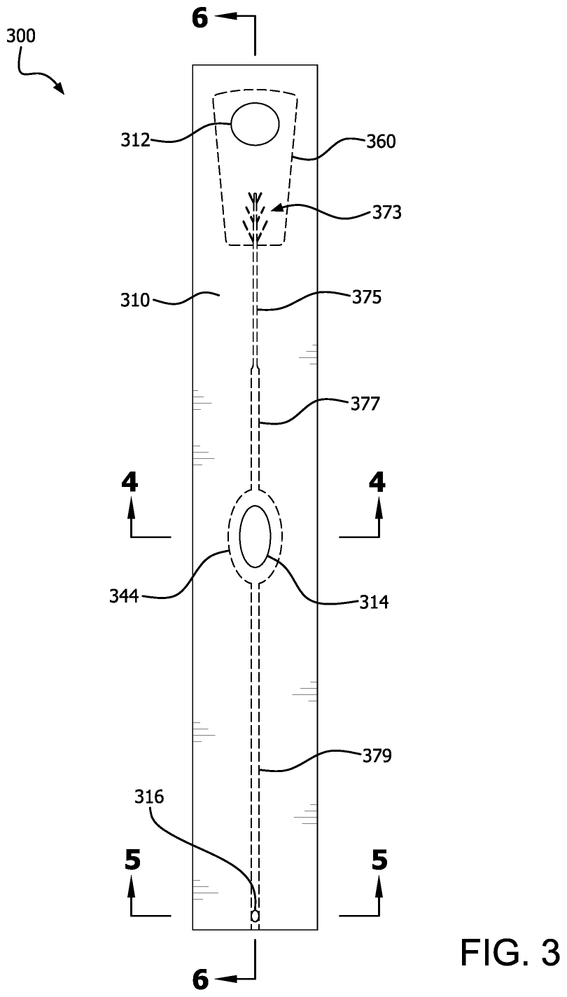

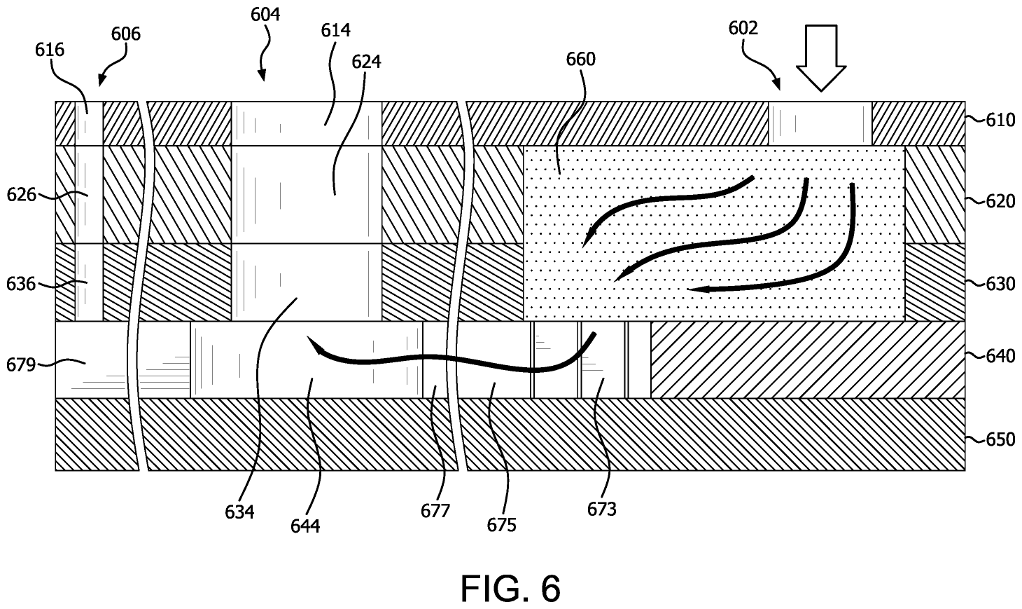

To aid discussion, each of the microfluidic capillary channels may be referred to as having a first end and a second end. So, for example, and as illustrated in FIG. 2, the first microfluidic capillary channel may comprise a narrow portion 275 having a first end and a second end, the first end of the narrow portion 275 is fluidically connected to the plurality of plasma collection channels 273 and the second end of the narrow portion 275 is fluidically connected to a first end of a wider portion 277 of the first microfluidic capillary channel. A second end of the wider portion 277 is fluidically connected to the aperture 244 that forms the lower portion of the plasma collection reservoir (104 in FIG. 1). As best seen in FIGS. 3 and 6, the plurality of plasma collection channels 273 is positioned under the engineered filter pad 260 but shifted off from the center of the engineered filter pad 260 to promote some lateral flow of plasma through the engineered filter pad 260.

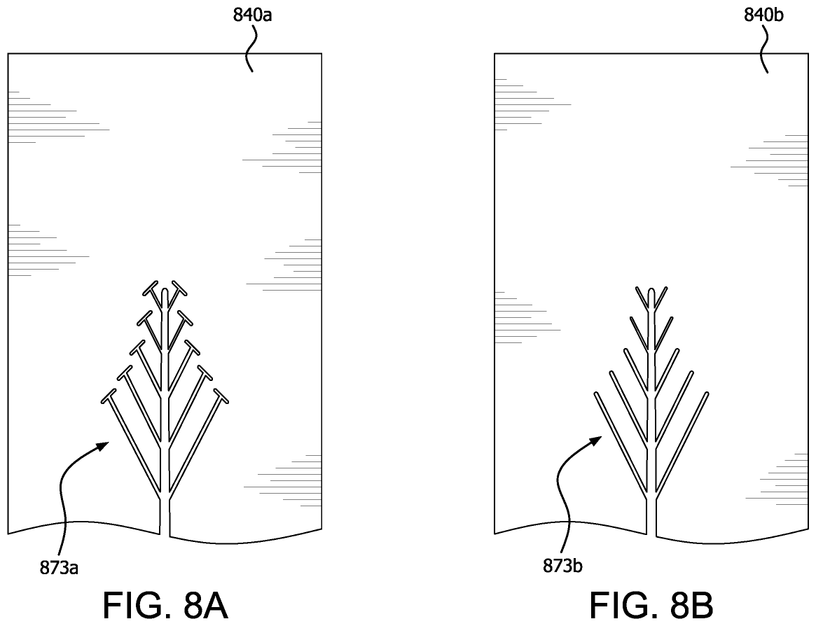

In various embodiments, and as best seen in FIGS. 7 and 8A-8B, the plurality of plasma collection channels 273 may be disposed in a dendritic configuration (like a dendritic drainage system or pattern) whereby each of the collection channels function as a contributing stream that join together into a single culminated channel that seamlessly becomes the first end of the narrow portion 275 of the first microfluidic capillary channel 275/277. In this way, the plasma collecting in each of the contributing collection channels of the dendritic system channels directly into the first end of the narrow portion 275 of the first microfluidic capillary channel.

In various embodiments, each of the plurality of plasma collection channels 273, the narrow portion 275 and the wider portion 277 of the first microfluidic capillary channel, the aperture 244, and the second microfluidic capillary channel 279, are cut all the way through the thickness of the fourth layer 240. The channels 275, 277 and 279 become true “capillaries” by being capped on the top by third layer 230 and on the bottom by fifth layer 250. In other words, the microfluidic capillary channels 275, 277 and 279 are “boxed-in” structures, having either a square or a rectangular cross-section depending on the width of the longitudinal cut versus the thickness of the fourth layer 240. The aperture 244 becomes the bottom of the plasma collection reservoir (104 in FIG. 1) by being sealed closed on the bottom by fifth layer 250.

In various embodiments, the narrow portion 275 of the first microfluidic capillary channel 275/277 leading to the aperture 244 is about 20 mil (0.02 inches, 0.5 mm) wide, by about 5 mil (0.005 inch, 0.127 mm) tall, which is necessarily the thickness of the fourth layer 240. In various embodiments, the length of the narrow portion 275 of the first microfluidic capillary channel 275/277 is about 0.64 inches (16 mm).

In various embodiments, the wide portion 277 of the first microfluidic capillary channel 275/277 leading to the aperture 244 is about 40 mil (0.04 inches, 1 mm) wide, by about 5 mil (0.005 inch, 0.127 mm) tall, which is necessarily the thickness of the fourth layer 240. In various embodiments, the length of the wide portion 277 of the first microfluidic capillary channel 275/277 is also about 0.64 inches (16 mm).

In various embodiments, the ratio of the length of the narrow portion 275 to the wide portion 277 in the first microfluidic capillary channel leading to the aperture 244 is about 1:1. The ratio of the width of the wide portion 277 to the width of the narrow portion 275 is 2:1. If this ratio is increased, such as up to about 5:1, the capillary draw is too slow to move plasma from the outlet of the engineered filter pad 260.

As illustrated in FIG. 2 (and also seen in FIG. 7), the narrow portion 275 transitions gradually into the wide portion 277, with a tapered transition, so as to reduce turbulence in the plasma flow. The narrow portion 275 aids in pulling the plasma quickly from the engineered filter pad 260, whereas the wide portion 277 slows the flow down. The use of an initial narrow portion 275 transitioning into a wide portion 277 for the first microfluidic capillary channel optimizes the speed of the plasma separation device without creating too large of a capillary draw on the engineered filter pad so as to pull blood cells and platelets from the filter.

In various embodiments, the second microfluidic capillary channel 279 is also about 40 mil (0.04 inches, 1 mm) wide, by the thickness of the fourth layer 240, which is about 5 mil (0.005 inch, 0.127 mm). The length of the second microfluidic capillary channel 279 leading from the aperture 244 to the air vent is about 1.8 inches (46 mm).

In various embodiments, the ratio of the length of the first microfluidic capillary channel 275/277 to the length of the second microfluidic capillary channel 279 is about 0.7:1.

The second microfluidic capillary channel 279, in combination with the air vent (106 in FIG. 1), the volume of which is the combination of the three apertures 216, 226 and 236, ensures a complete fill of the plasma collection reservoir (104 in FIG. 1) due to an increase in capillary pull from this structure. The fact that the second microfluidic capillary channel 279 will eventually fill with separated plasma requires that the length of the second microfluidic capillary channel 279 be optimized to this length and ratio to the length of the first microfluidic capillary channel 275/277.

As mentioned, and with reference to FIG. 2, the aperture 244 dimensionally defines the bottom portion of the plasma collection reservoir (104 in FIG. 1). The longer axis of the oval shaped aperture is about 0.5 inches (13 mm). The aperture 244 in combination with the apertures 214, 224 and 234 define the total capacity of the plasma collection reservoir (104 in FIG. 1). A target volume for the plasma collection reservoir (104 in FIG. 1) depends at least in part to the intended use of the plasma separation device 200, and whether it is directly coupled to a diagnostic device. The dimensions discussed above in the context of FIG. 2 are optimized for separation and collection of plasma from a droplet sized blood sample applied at the blood application port (102 in FIG. 1), wherein the separated plasma will not entirely fill the reservoir, and it can be removed by a pipette inserted into the plasma collection reservoir.

In various embodiments, the fourth layer 240 (the “channel layer”) may further comprise a single layer of uniform material or a laminated structure of at least two layers. In various embodiments, the fourth layer 240 may comprise a three layer laminate further comprising an adhesive tape layer, such as 3M™ 9969 Transfer Adhesive Tape (from 3M), which is a 1 mil (0.001 inch, 0.0354 mm) thick layer of pressure sensitive neutral acrylic resin, adhered to a layer of ARflow® 93049 (from Adhesives Research), which is a 3 mil (0.003 inch, 0.77 mm) flexible polyester plastic film coated on one side with a hydrophilic pressure-sensitive adhesive. With this construction, the fourth layer 240 comprises a core polyester film layer of about 3 mil (0.003 inch, 0.77 mm) thickness, having a 1 mil (0.001 inch, 0.0354 mm) thick hydrophilic adhesive on one side, and a 1 mil (0.001 inch, 0.0354 mm) thick adhesive tape on the opposite side. The combination of the adhesive tape, polyester film core and adhesive coating provides the 5 mil (0.005 inch, 0.127 mm) thickness mentioned for the fourth layer 240. The fourth layer 240, being rotationally symmetrical about the longitudinal axis other than the nature of the adhesive on opposite sides (which may be the same or chemically different) can be oriented either way between the third layer 230 and the fifth layer 250 such that the third layer 230, fourth layer 240 and fifth layer 250 are bonded together.

With continued reference to FIG. 2, the microfluidic passive plasma separation device 200 comprises a fifth layer 250, disposed at the bottom of the five layer laminate structure. Like the top layer 210, the bottom layer comprises a non-porous plastic film, but absent any apertures. In this way, the fifth layer 250 seals off the bottom of the microfluidic passive plasma separation device 200. In various embodiments, the fifth layer 250 can comprise any type of plastic film, such as 4 mill (0.004 inch, 0.1 mm) polyester. The polyester film may comprise a hydrophilic treatment on at least the side put into contact with the channel layer, fourth layer 240, because the fifth layer 250 forms the bottom of the capillary channels discussed as having a boxed-in structure. In various embodiments, the fifth layer 250 comprises a 4 mil (0.004 inch, 0.1 mm) thick clear hydrophilic polyester film, such as 3M™ 9969 Microfluidic Diagnostic Film available from 3M.

In various embodiments, the five layers depicted in FIG. 2 may be laminated together, simply by pressure rollers since the adhesives in the various layers will bond the layers together. Heat and pressure can be used to form the microfluidic passive plasma separation device 200 from the stack of layers and the engineered filter pad seated therein.

FIG. 3 illustrates a top plan view of a microfluidic passive plasma separation device 300, in accordance with various embodiments of the present disclosure. Visible from the top of the device 300 is the first layer 310 of the multilayer laminated structure. The first layer 310 further comprises the aperture 312 that forms part of the blood application port (102 in FIG. 1), the aperture 314 that forms part of the plasma collection reservoir (104 in FIG. 1), and the aperture 316 that forms part of the air vent (106 in FIG. 1). The dashed lines represent the features that are hidden from view inside the device, including the engineered filter pad 360, the dendritic configured plurality of plasma collection channels 373 under the engineered filter pad 360, the narrow portion 375 and wide portion 377 that together form the first microfluidic capillary channel (375/377) that fluidically connects to the plurality of plasma collection channels 373, the larger sized aperture 344 configured in the fourth layer (240 in FIG. 2) that forms part of the plasma collection reservoir and that is fluidically connected to the first microfluidic capillary channel (375/377), and the second microfluidic capillary channel 379 that extends a fluidic connection from the aperture 344 to the air vent (106 in FIG. 1). In this way, each of these elements, from the engineered filter pad 360 through to the air vent aperture 316, are in fluidic communication.

With continued reference to FIG. 3, the dendritic structure of channels 373 is purposely off-center from being directly underneath the engineered filter pad 360. The plurality of channels is vertically offset from the blood application port (shown in location by the aperture 312 in the first layer 310), such that the inlet to the engineered filter pad 373 is laterally displaced from the plurality of plasma collection channels under the filter pad 360. This configuration forces the blood sample applied at the port 312 to move vertically and laterally down through the engineered filter pad 360 before dropping into the various individual collection branches of the dendritic collection structure. This longer route through the engineered filter pad 360 increases the separation efficiency. The dendritic structure ensures there is sufficient pull on the outlet of the engineered filter pad 360. Dendritic structures for the plurality of plasma collection channels are discussed in reference to FIGS. 7, 8A and 8B.

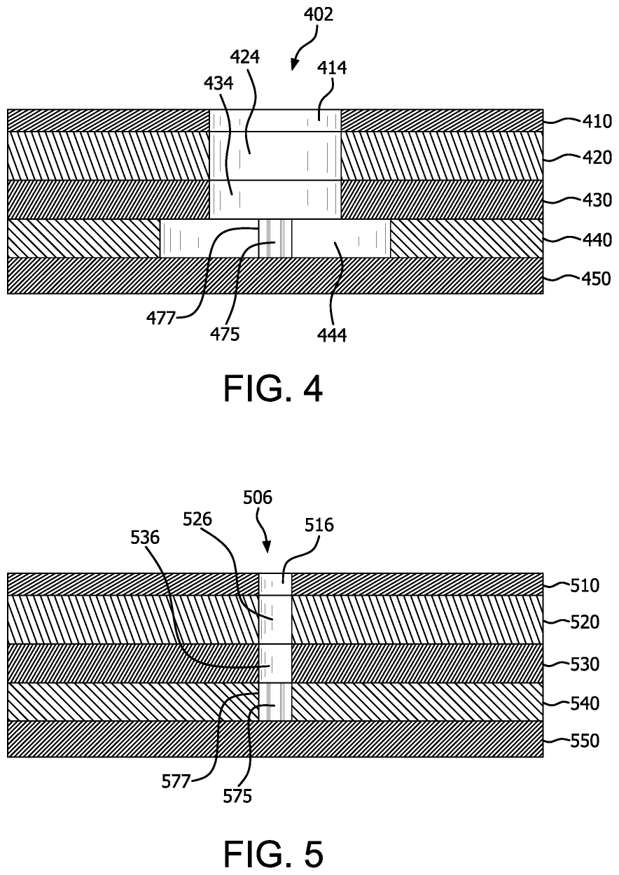

With further reference to FIG. 3, the location of three separate cross-sections are shown that form the basis for the next three drawing figures. Cross-section 4-4 is taken laterally through the plasma collection reservoir and viewed toward the inlet end of the device (FIG. 4). Cross-section 5-5 is taken laterally through the air vent and viewed toward the inlet end of the device (FIG. 5). Lastly, cross-section 6-6 is taken longitudinally down the center axis of the device (FIG. 6), with the view either direction being the same other than the two views being mirror images.

FIG. 4 is the 4-4 cross-sectional view as indicated in FIG. 3. FIG. 4 shows the plasma collection reservoir 402 in cross-section down the shorter (lateral) axis of its oval shape. As described previously, the volume of the plasma collection reservoir 402 is defined by the size of the apertures 414, 424, 434 and 444 disposed in each of the first layer 410, second layer 420, third layer 430 and fourth layer 440. The configuration having the larger oval aperture 444 at the bottom is purposely done to provide a larger volume for plasma toward the bottom of the reservoir, ensuring that the reservoir will not fill entirely. Looking in this direction in the cross-section, the wider portion 477 of the first microfluidic capillary channel is visible, which is seen to neck down into the narrow portion 475 of the first microfluidic capillary channel. The plurality of plasma collection channels, although not visible, are at the end of this channel. As shown in cross-section, microfluidic passive plasma separation device comprises the fifth layer 450 that seals off the bottom of the device in that the layer does not include any apertures.

FIG. 5 is the 5-5 cross-sectional view as indicated in FIG. 3. FIG. 5 shows the air vent 506 for the microfluidic passive plasma separation device. As shown, the air vent 506 comprises a combination of aperture 516 in the first layer 510, aperture 526 in the second layer 520, aperture 536 in the third layer 530 and part of the second microfluidic capillary channel 579. Looking in this direction in the cross-section, the second microfluidic capillary channel 579 is visible, which is seen to neck down, on the other side of the aperture disposed in the fourth layer 440 as part of the plasma collection reservoir, into the narrow portion 575 of the first microfluidic capillary channel before reaching the plurality of plasma collection channels at the end of this channel (not visible). As shown in cross-section, microfluidic passive plasma separation device comprises the fifth layer 550 that seals off the bottom of the device in that the layer does not include any apertures.

FIG. 6 illustrates the 6-6 cross-sectional view indicated in FIG. 3. This cross-section is a longitudinal cross-section through the entire length of the microfluidic passive plasma separation device. The two “wavy” double lines through the illustration are to shorten redundant lengths of the device that would cause the illustration not to fit on a page. FIG. 6 also shows plasma flow with bolded curving arrows. The device comprises a blood application port 602 wherein a sample of whole blood is placed (as per the block arrow). In various embodiments, the blood application port 602 comprises just the single aperture shown disposed through the first layer 610. The engineered filter pad 660 is located beneath the blood application port 602, and fixed in place laterally by the apertures configured in the second layer 620 and the third layer 630, and boxed-in by the first layer 610 and the fourth layer 640. The whole blood sample moves laterally downwards through the engineered filter pad 660 as indicated by the flow arrows as a consequent of the offset in location of the blood application port 602 and the plurality of plasma collection channels 673 configured underneath the engineered filter pad 660. As described herein, the filter pad separates the plasma, but it is the capillary network comprising each of the features in fluidic communication, that operate to pull the plasma through by capillary action so that the filter pad 660 separates the plasma faster than if operating only by gravity. The fourth layer 640 of the device comprises the plurality of plasma collection channels 673, the initial narrow portion 675 of the first microfluidic capillary channel, the wide portion 677, the oval aperture 644 that form part of the plasma collection reservoir 604, and the second microfluidic capillary channel 679 fluidically leading to and ending in the air vent 606. The plasma collection reservoir 604 is a combination of the apertures 614, 624, 634 and 644 disposed in layers 610, 620, 630 and 640 respectively. The air vent 606 is a combination of the apertures 616, 626 and 636 disposed in layers 610, 620 and 630, respectively.

As illustrated in FIG. 6, separated plasma is pulled by capillary action into the plurality of plasma collection channels 673, into the narrow portion 675 and wide portion 677 of the first microfluidic capillary channel 675/677, and into the aperture 644 of the plasma collection reservoir. How high the collected plasma ends up in the plasma collection reservoir 604 depends in part on the size of the blood sample placed at the blood application port 602.

In general, the plurality of plasma collection channels disposed underneath the engineered filter pad coax the plasma to leave the outlet of the engineered filter pad, creating droplets of plasma that converge in the main trunk of the plurality of collection channels and into the first microfluidic capillary channel. Once the droplets of plasma begin to fall into the dendritic structure and into the first microfluidic capillary channel, capillary action drives the plasma forward through the channel creating a drop in pressure at the outlet of the filter pad that coaxes even more plasma out from the filter pad, creating a stream that will eventually fill the first microfluidic capillary channel and part of the plasma collection reservoir. Once the collection reservoir and the first and second microfluidic capillary channels are each filled with separated plasma, the pressure of the capillary pump is alleviated, thereby shutting off the stream. This self-regulation, i.e., the ability for the device to shut itself off, prevents blood cells from being pulled out from the engineered filter pad and into the reservoir, compromising the quality of the separated plasma.

In various embodiments, the combined volume of the plurality of plasma collection channels, the first microfluidic capillary channel and the plasma collection reservoir, is from about 25 μL to about 35 μL. In various embodiments, the combined volume of the plurality of plasma collection channels, the first microfluidic capillary channel and the plasma collection reservoir is about 32 μL.

In various embodiments, a microfluidic passive plasma separation device herein provides optically clear plasma, and is capable of handling an initial whole blood sample having a volume of from about 100 μL to about 150 μL. In various versions of the device, an initial whole blood sample has a volume of about 120 μL. In various experiments, a device according to the present disclosure will capture about 20 μL to about 25 μL optically pure plasma from a whole blood sample measuring from about 100 μL to about 150 μL. In various embodiments, about 24 μL optically clear plasma is obtained from a whole blood sample measuring about 120 μL in a timespan of about 60 to about 90 seconds. In various embodiments, a microfluidic passive plasma separation device herein can provide initial separation of plasma in about 15 seconds, about 50% by volume separation of the blood sample in about 30 seconds, and greater than about 85% by volume separation of the blood sample in about 45 seconds. In various embodiments, the separated plasma is optically clear, with little to no hemolysis.

The cross-sectional view of the device in FIG. 6 helps illustrate how the device is able to shut itself off and thus be self-limiting. The plurality of plasma collection channels 673, the first and second microfluidic capillary channels 675, 677 and 679, the plasma collection reservoir 604, and the air vent 606, collectively provide a capillary pump sufficient to draw plasma through the filter pad 660 and pump it, under capillary pressure, into the plasma collection reservoir. In other words, the capillary action pulls the blood through the engineered filter pad 660 in the same way an electromechanical pump might. The directional arrows in the illustration show the direction of capillary flow of plasma. For the device to shut itself off, the hydrostatic head of pressure resulting from the vertical column of liquid plasma collected in the plasma collection reservoir equals the capillary pressure in the device. That is, the resulting hydrostatic head pressure in the plasma collection reservoir equals the force that had been drawing the plasma down the first microfluidic capillary channel and into the reservoir. The capillary pump is not capable of pushing plasma against the hydrostatic head created in the plasma collection reservoir 604. Thus, the combination of downward flow, lateral flow and upward pooling of plasma is critical for the device to have the ability to shut itself off. More specifically, the height of the plasma collection reservoir 604 has to be greater than the height of the first microfluidic capillary channel (defined by the combined thicknesses of the layers 610 through 640), and the outlet of the engineered filter pad 660 (positioned in the third level 630), need to be higher than the first microfluidic capillary channel (which is configured in the fourth level 640). As illustrated in FIG. 6, these aspects are assured by disposing the various features (position of the engineered filter pad, the various channels and apertures) in multiple layers of a laminated structure such that there are combinations of lateral and vertical capillary flow and an assurance that a hydrostatic head pressure will develop in the plasma collection reservoir 604 from the vertical column of plasma.

As mentioned, a continuous draw of material through the engineered filter pad 660 can lead to contamination of the collected plasma as cells and platelets become dislodged from the filter pad 660. The variables that may be optimized so that both the separation rate and the purity of the separated plasma are maximized, include, but are not limited to, the volume and dimensions of the first and second microfluidic capillary channels, including the ratio of the lengths of the narrow and wide portions of the first microfluidic capillary channel, which in turn determines the strength of the capillary pump and the extent of pull of plasma out from the outlet side of the filter pad 660, and the volumetric capacity and shape of the plasma collection reservoir 604 to control the length of time the device operates with a single sample of whole blood placed at the blood application port 602. In various embodiments,

FIG. 7 is an enlarged view of the fourth layer 240 in FIG. 2, indicated by the dashed line boundary. This enlargement shows the details of embodiments of the plurality of plasma collection channels 773, and the narrow portion 775 and wide portion 777 of the first microfluidic capillary channel, disposed through the thickness of the fourth layer 710 of the device. As mentioned, in various embodiments, the plurality of plasma collection channels 773 comprises a dendritic structure such that the collection channels can converge into the narrow portion 775 of the first microfluidic capillary channel. Various dimensions are indicated in the illustration, namely, D1 through D7.

In various embodiments, a plurality of plasma collection channels configured as a dendritic structure comprises individual collection branches that each converge into a main collection branch. In various embodiments, the number of individual collection branches in a plurality of plasma collection channels is from about two (2) to about ten (10) or more, depending on the nature and dimensions of the engineered filter pad, the dimensions of the first microfluidic capillary channel, and other variables.

In FIG. 7, D1 represents the distance from the beginning of the dendritic structure 773 to the transition from the narrow portion 775 to the wide portion 777 of the first microfluidic capillary channel. In various embodiments, this distance D1 is from about 0.5 inches (12.7 mm) to about 1.5 inches (38 mm). D2 represents the distance from the transition of the narrow portion 775 and the wide portion 777 of the first microfluidic capillary channel all the way to the end of the device, recognizing that D1 and D2 together form the length of the entire capillary system. In various embodiments, this distance D2 is from about 2.5 inches (63.5 mm) to about 3.5 inches (89 mm). In various embodiments, D1 is about 0.9 inches (23 mm) and D2 is about 2.92 inches (74 mm), making the ratio of D2/D1 about 3:1.

The distances D3 and D4 are relevant to the dendritic structure 773. The main branch of the structure has a width D3 of about 0.01 inches (0.254 mm), whereas an individual collection branch has a width D4 of only about 0.0035 inches (0.09 mm). 0.0035 inches (0.09 mm) is about the physical limits of a CO₂ laser, so it's not likely these channels can be formed any smaller. In various embodiments, the dendritic structure 773 comprises a single main channel and six (6) collection channels such that the structure comprises a symmetrical tree structure. Other configurations of use herein will be discussed in reference to FIGS. 8A and 8B.

D5 and D6 are widths of the two portions of the first microfluidic capillary channel that have already been discussed. In preferred embodiments it was noted that the width of the narrow portion 775 may be about 0.02 inches (0.5 mm) whereas the width of the wide portion 777 may be about 0.04 inches (1 mm), such that the ratio of widths is about 1:2. Channels of these widths may be stamped with a die rather than laser cut through the fourth layer 740. D7 represents the thickness of the fourth layer 710, which in preferred embodiments is about 5 mil (0.005 inch, 0.127 mm) thick.

FIGS. 8A and 8B illustrate optional configurations for the plurality of plasma collection channels 873a and 873b, respectively, which also find use in the plasma separation devices of the present disclosure. Both of these configurations are dendritic structures. In FIG. 8A, the dendritic structure 873a comprises ten (10) collection channels, each with a “T” shape. In FIG. 8B, the dendritic structure 873b comprises ten (10) collection channels, each having a straight channel shape per the structures in the previous drawing figures. As mentioned, there is a balance between the number of collection branches in a dendritic structure and the pull from the outlet of the engineered filter pad. Configurations as per FIGS. 7, 8A and 8B are ideal configurations in that they provide sufficient capillary pull without liberating blood cells and platelets from the engineered filter pad. The widths of the branches are about as narrow as possible, so the only other practical variable is increasing or decreasing the number of branches in the dendritic system. In various embodiments, the number of collection branches in a plurality of plasma collection channels is from about two (2) to about ten (10), wherein each collection branch has a width of about 0.0035 inches (0.09 mm) and a height of about 0.005 inch (0.127 mm). The individual collection branches, like a tree, converge into a single main collection branch or channel, and the main collection branch is fluidically connected to the first microfluidic capillary channel.

### Methods of Construction

In various embodiments, a microfluidic passive plasma separation device herein may comprise a single block of material or a plurality of layers that are bonded together by virtue of adhesive coatings or adhesive tape layers, or by a pressure and/or heated, or sonic welding lamination process that melts and bonds the layers together. If machining a single block of material or disposing features in one or more layers used in a multilayer laminate construct, the various plasma collection channels, microfluidic capillary channels and apertures are formed by removal of material. Removal of material can comprise laser cutting and ablation, or die stamping, including ballistic punching, depending on the dimensions of the feature needed in the material.

For example, apertures and troughs in a layer of a multilayer construct herein may have dimensions equal to or greater than about 0.01 inch (0.254 mm), and are therefore large enough to be die stamped. The stamping may comprise standard stamping methods or the higher speed and higher momentum version referred to as ballistic punching. The stamping process is best used for forming apertures and channels that go completely through a layer, whereas laser ablation can be used to remove material when machining a block of material or removing some material from a surface without going all the way through the material. For example, a main collection channel within a plurality of plasma collection channels having a dendritic structure may have a width of about 0.01 inches (0.254 mm), which can be stamped through. The smaller dimensioned features, i.e., less than about 0.01 inch (0.254 mm), particularly the individual branches within the plurality of plasma collection channels, are best formed by laser ablation of material. For example, individual collection branches in the dendritic structure of plasma collections channels may have widths of only about 0.0035 inches (0.09 mm), which is about the physical limits of a CO₂ laser. These ultrathin channels cannot be stamped.

In various embodiments, a laser may be used to bore one or more longitudinally oriented microfluidic capillary channels through a length of a single block of material or through one or more layers of a multilayered device, or to cut one or more longitudinal troughs down a single layer in a desired length. In other examples, a laser may be used to punch features through a layer to accommodate vertical flow of fluid. Other means, e.g., chemical etching, may be used to create channels.

### Integrated Diagnostics

In various embodiments, the pipette-accessible plasma collection reservoir is eliminated and the end of the first microfluidic capillary channel may be fluidically connected to the inlet of a POC or another diagnostic device. In this integrated configuration, a drawn sample of whole blood is placed on the blood application port to enter the inlet of the engineered filter pad, and the separated plasma flows directly into a diagnostic device where it can be analyzed.

### Method for Separating Plasma from Whole Blood

In various embodiments, a microfluidic passive plasma separation device in accordance with the present disclosure is used in a method for separating plasma from a sample of whole blood. In various embodiments, a method for separating optically clear plasma from a sample of whole blood is described. The method comprises disposing a whole blood sample at an inlet of an engineered filter pad having an inlet and outlet and capable of providing separated plasma at the outlet from whole blood disposed at the inlet; and conveying the separated plasma by capillary action through at least one microfluidic capillary channel to a plasma collection reservoir, wherein the microfluidic capillary channel comprises a first end and second end, the first end in fluidic communication with the outlet of the engineered filter pad, and the second end in fluidic communication with the plasma collection reservoir. In various embodiments, multiple microfluidic capillary channels in fluidic communication form a route for the separated plasma to travel from the outlet of the engineered filter pad to the plasma collection reservoir via capillary action. In various embodiments, the route comprises both lateral and vertical flow of the separate plasma from the outlet of the engineered filter pad to the plasma collection reservoir. At the end of the separation event, a hydrostatic head in the plasma collection reservoir meets or exceeds the capillary pressure to self-limit the device.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A multilayered microfluidic device comprising:
an engineered filter pad having an inlet and an outlet, the engineered filter pad capable of separating plasma from whole blood;
a plurality of plasma collection channels in fluidic communication with the outlet of the engineered filter pad;
a first microfluidic capillary channel having a first end and a second end, the first end of the first microfluidic capillary channel in fluidic communication with the plurality of plasma collection channels; and
a plasma collection reservoir in fluidic communication with the second end of the first microfluidic capillary channel, the plasma collection reservoir configured to collect separated plasma;
wherein the plurality of plasma collection channels, the first microfluidic capillary channel and a portion of the plasma collection reservoir are configured in a single layer of the multilayered microfluidic device.

2. The multilayered microfluidic device of claim 1, further comprising a blood application port.

3. The multilayered microfluidic device of claim 2, wherein the blood application port and the engineered filter pad are configured on separate layers of the multilayered microfluidic device.

4. The multilayered microfluidic device of claim 2, wherein the blood application port is configured in a first layer, and wherein the engineered filter pad is positioned in a second layer adjacent the first layer such that the blood application port is in fluidic communication with the inlet of the engineered filter pad.

5. The multilayered microfluidic device of claim 2, wherein the blood application port is configured on a layer distinct from the single layer containing the plurality of plasma collection channels, the first microfluidic capillary channel, and a portion of the plasma collection reservoir.

6. The multilayered microfluidic device of claim 2, wherein the engineered filter pad is disposed between the blood application port and the plurality of plasma collection channels, and is in fluidic communication with the blood application port and the plurality of plasma collection channels.

7. The multilayered microfluidic device of claim 1, wherein the inlet of the engineered filter pad and the plurality of plasma collection channels are spatially arranged in the multilayered microfluidic passive plasma separation device to promote both lateral and vertical flow of a blood sample through the engineered filter pad.

8. The multilayered microfluidic device of claim 1, wherein the first microfluidic capillary channel comprises a narrow portion having a first length and a first width that transitions into a wide portion having a second length and a second width, the narrow portion in fluidic communication with the plurality of plasma collection channels and the wide portion in fluidic communication with the plasma collection reservoir.

9. The multilayered microfluidic device of claim 1, wherein the plurality of plasma collection channels, the first microfluidic capillary channel, and the plasma collection reservoir are each configured to collectively provide a capillary pump sufficient to pull separated plasma through the engineered filter pad and convey the separated plasma from the outlet of the engineered filter pad to the plasma collection reservoir.

10. The multilayered microfluidic device of claim 9, wherein a portion of the plasma collection reservoir and the outlet of the engineered filter pad are spatially configured above the first microfluidic capillary channel such that plasma collected in the plasma collection reservoir can provide a sufficient hydrostatic head pressure to overcome the capillary pump.

11. The multilayered microfluidic device of claim 1, further comprising an air vent fluidically connected to the plasma collection reservoir by a second microfluidic capillary channel such that the outlet of the engineered filter pad, the plurality of plasma collection channels, the first microfluidic capillary channel, the plasma collection reservoir, the second microfluidic capillary channel, and the air vent are fluidically connected to one another in series.

12. The multilayered microfluidic device of claim 11, wherein the plurality of plasma collection channels, the first microfluidic capillary channel, the plasma collection reservoir, the second microfluidic capillary channel, and the air vent are each configured to collectively provide a capillary pump sufficient to pull separated plasma through the engineered filter pad and convey the separated plasma from the outlet of the engineered filter pad to the plasma collection reservoir.

13. The multilayered microfluidic device of claim 12, wherein a portion of the plasma collection reservoir and the outlet of the engineered filter pad are spatially configured above the first microfluidic capillary channel such that plasma collected in the plasma collection reservoir can provide a sufficient hydrostatic head pressure to overcome the capillary pump.

14. The multilayered microfluidic device of claim 1, wherein the plurality of plasma collection channels comprises a dendritic structure further comprising a plurality of branches and a single main channel, wherein each branch converges into the single main channel that fluidically connects to the first end of the first microfluidic capillary channel.

15. The multilayered microfluidic device of claim 1, wherein the engineered filter pad comprises a homogeneous matrix of borosilicate glass microfibers coated with an agglutinating agent.

16. The multilayered microfluidic device of claim 1, wherein the engineered filter pad has an average pore size of 0.006 mm (6 μm) to about 0.01 mm (10 μm).

17. A method for separating plasma from a blood sample in a multilayered microfluidic device, the method comprising:
disposing a blood sample at an inlet of an engineered filter pad having an inlet and outlet, the engineered filter pad configured to provide separated plasma at the outlet from a blood sample disposed at the inlet;
separating plasma through the engineered filter pad to the outlet of the engineered filter pad;
pulling the separated plasma from the outlet of the engineered filter pad into a plurality of plasma collection channels and conveying the separated plasma from the plurality of collection channels through a microfluidic capillary channel to a plasma collection reservoir by capillary forces; and
collecting the separated plasma as a liquid in the plasma collection reservoir;
wherein the plurality of plasma collection channels is in fluidic communication with the outlet of the engineered filter pad, the microfluidic capillary channel comprises a first end and a second end, the first end of the microfluidic capillary channel is in fluidic communication with the plurality of plasma collection channels and the second end of the microfluidic capillary channel is in fluidic communication with the plasma collection reservoir, and wherein the plurality of plasma collection channels, the microfluidic capillary channel and a portion of the plasma collection reservoir are configured within a single layer of the multilayered microfluidic device.

18. The method of claim 17, wherein the plurality of plasma collection channels, the microfluidic capillary channel, and the plasma collection reservoir are each configured to collectively provide a capillary pump sufficient to pull separated plasma through the engineered filter pad and convey the separated plasma from the outlet of the engineered filter pad to the plasma collection reservoir by the capillary forces.

19. The method of claim 18, wherein a portion of the plasma collection reservoir and the outlet of the engineered filter pad are spatially configured above the microfluidic capillary channel such that the separated plasma collected in the plasma collection reservoir provides a hydrostatic head of sufficient pressure to overcome the capillary forces and cease plasma separation through the engineered filter pad.

20. The method of claim 17, wherein about 24 μL of optically clear plasma is obtained from an initial whole blood sample measuring about 120 μL, in the course of about 60 to about 90 seconds.
